(19) United States
(12) Patent Application Publication
(10) Pub. No.: US 2023/0273227 A1
(43) Pub. Date: Aug. 31, 2023

(54) METHOD FOR TESTING DIFFERENT HORMONES BASED ON DIFFERENT TIME PERIODS OF PREGNANCY PREPARATION

(71) Applicant: Lollipop Technology (Hangzhou) Co., Ltd., Hangzhou (CN)

(72) Inventors: Lei LU, Hangzhou (CN); Hongqun LI, Hangzhou (CN)

(21) Appl. No.: 17/660,211

(22) Filed: Apr. 22, 2022

(30) Foreign Application Priority Data
Feb. 25, 2022 (CN) 202210182415.X
Feb. 25, 2022 (CN) 202210182983.X
Feb. 25, 2022 (CN) 202220405968.2

(51) Int. Cl. G01N 33/76; G01N 33/543

(52) U.S. Cl. CPC G01N 33/76; G01N 33/54387

(57) ABSTRACT

The present invention discloses a method for testing different hormones based on different time periods of pregnancy preparation, including four method steps: fertility test, ovulation test, ovulation confirmation test and pregnancy test. The present invention provides a set of systematic and perfect pregnancy preparation management plan for pregnancy preparation women, which can provide reasonable and effective test guidance for pregnancy preparation women, helps pregnancy preparation women to determine and analyze various hormone test results, and gives corresponding suggestions based on the test results to better help women complete the pregnancy preparation process. The present invention comprehensively and systematically analyzes and determines various hormone levels of women during pregnancy preparation, with high accuracy and reliable analysis results. This pregnancy preparation management method, combined with a hormone test device, enables pregnancy preparation women to complete various hormone tests at home and analyze the test results without going to the hospital frequently, has little impact on life, and brings convenience to the pregnancy preparation women.

### CROSS-REFERENCE TO RELATED APPLICATIONS

This application claims the benefit and priority of Chinese Patent Application Number 202220405968.2, 202210182983.X, 202210182415.X, which filed on Feb. 25, 2022 with China National Intellectual Property Administration, the disclosures of which are incorporated herein by reference in their entireties.

### BACKGROUND OF THE PRESENT INVENTION

### Field of Invention

The present invention relates to the technical field of women's pregnancy preparation period management, in particular to a method for testing different hormones based on different time periods of pregnancy preparation.

### Description of Related Arts

Women's pregnancy preparation period management usually means that women determine the best date of conception by tracking hormones or basal body temperature data in the menstrual period, so as to increase the probability of conception.

Hormone test items involved in the women's pregnancy preparation period management mainly include the following: 1, fertility test; 2, ovulation test; 3, ovulation confirmation; and 4, pregnancy test.

Now pregnancy preparation women usually buy hormone test devices from the market to test various hormone (follicle-stimulating hormone (FSH), luteinizing hormone (LH), pregnanediol-3-glucuronide (PDG), human chorionic gonadotropin (HCG), etc.) levels in the body. Although the pregnancy preparation women can test various hormone levels in the body with the hormone test devices, most of them lack professional knowledge and cannot make reasonable and effective hormone test management plans. Meanwhile, they cannot comprehensively evaluate the status of pregnancy preparation through the test data or obtain reasonable suggestions for pregnancy preparation from the data results.

Although hospitals and laboratories can provide more accurate full test services, the procedures are cumbersome and the prices are expensive in the hospital. On the other hand, the results of the tests done in the hospital usually cannot be provided in time, which is not beneficial to high-frequency test and tracking (generally, significant changes of the above hormone levels occur in women's menstrual period, and high-frequency test can reflect the changing trend of the above hormones in the menstrual period).

The objective of the present invention is to provide a method for testing different hormones based on different time periods of pregnancy preparation. When used with a hormone test device, the method can formulate a reasonable and effective hormone test management plan for pregnancy preparation women, help the pregnancy preparation women to analyze the hormone test results, and give corresponding suggestions based on the test results to better help women complete the pregnancy preparation process.

### SUMMARY OF THE PRESENT INVENTION

The objective of the present invention is to provide a method for testing different hormones based on different time periods of pregnancy preparation. When used with a hormone test device, the method can formulate a reasonable and effective hormone test management plan for pregnancy preparation women, help the pregnancy preparation women to analyze the hormone test results, and give corresponding suggestions based on the test results to better help women complete the pregnancy preparation process.

The objective of the present invention is achieved by the following technical solutions: a method for testing different hormones based on different time periods of pregnancy preparation, including the following steps:
1) fertility test: on day 3 of a menstrual period, collecting a subject's urine as a test sample, testing the content of follicle-stimulating hormone in the test sample with a hormone test device, and when the content of follicle-stimulating hormone in the urine is more than 15 mIU/mL, prompting damage of the ovarian function or menopause;
2) ovulation test: on day 10-12 of the menstrual period, collecting the subject's urine as a test sample, testing the content of luteinizing hormone in the test sample with a hormone test device, and if the tested content does not reach 10 mIU/mL or more, testing the content of luteinizing hormone again at the same time the next day until the tested content reaches 10 mIU/mL or more; then testing the content of luteinizing hormone once every 4-6 hours until the tested content reaches 35 mIU/mL or more, and based on the time period of 24-48 hours after the current time as a predicted first ovulation period, prompting the subject to conceive within the first ovulation period; continuing to test the content of luteinizing hormone in the urine every 4-6 hours after the first ovulation period until the tested content is restored to any value below 35 mIU/mL, then arriving at a second ovulation time, prompting the subject to conceive within the second ovulation period;
3) ovulation confirmation test: on at least three consecutive days within 7-days after ovulation, collecting the subject's urine as a test sample, testing the content of pregnanediol-3-glucuronide in the test sample with a hormone test device, and if the contents of pregnanediol-3-glucuronide on any two consecutive days within 7-10 days after ovulation exceed 5 μg/mL, confirming that the subject has successfully ovulated during the ovulation period; and
4) pregnancy test: on day 10-14 after the first ovulation time, testing the content of human chorionic gonadotropin in a urine sample with a hormone test device, and when the content tested on any day of day 10-14 after the first ovulation time reaches or exceeds 10 mIU/mL, determining that the subject is pregnant.

Preferably, the hormone test device shown includes a test strip, the test strip includes test paper, and the test paper is provided with a sample adding zone, a marker zone, and a reaction zone; the marker zone contains one or more specific binding substances a which are marked by markers and can specifically bind to biochemical substances to be tested, the reaction zone is provided with test lines corresponding to the biochemical substances to be tested, and each test line is at least immobilized with one of a corresponding substance of the same type as the biochemical substance to be tested or a specific binding substance b that can specifically bind to the biochemical substance to be tested.

Preferably, the reaction zone is further provided with quality control lines, and each quality control line is quantitatively immobilized with a specific binding substance c capable of binding and capturing the specific binding substance a.

Preferably, the test paper is further provided with an absorption zone. Preferably, the marker is colloidal gold or a fluorescent marker.

Preferably, the hormone test device further includes an instrument body, and the instrument body is provided with a display screen, a circuit module, and a light path detection module capable of performing light detection on the reaction zone of the test paper.

Preferably, the biochemical substance to be tested is one of or a combination of luteinizing hormone, pregnanediol-3-glucuronide, follicle-stimulating hormone and human chorionic gonadotropin.

Preferably, the test strip is provided with a chip for storing test item information, and the instrument body is provided with an identification module for identifying and reading the chip.

The beneficial effects of the present invention are: the present invention provides a set of systematic and perfect pregnancy preparation management plan for pregnancy preparation women, which can provide reasonable and effective test guidance for pregnancy preparation women, helps pregnancy preparation women to determine and analyze various hormone test results, and gives corresponding suggestions based on the test results to better help women complete the pregnancy preparation process. The present invention comprehensively and systematically analyzes and determines various hormone levels of women during pregnancy preparation, with high accuracy and reliable analysis results. This pregnancy preparation management method, combined with the hormone test device, enables pregnancy preparation women to complete various hormone tests at home and analyze the test results without going to the hospital frequently, has little impact on life, and brings convenience to the pregnancy preparation women.

### BRIEF DESCRIPTION OF THE DRAWINGS

FIG. 1 is a flowchart of a method in the present invention.

FIG. 2 is a schematic structural illustration of a hormone test device in the present invention.

FIG. 3 is an exploded view of a test strip.

FIG. 4 is a schematic structural illustration of test paper.

FIG. 5 is an exploded view of an instrument body.

FIG. 6 is a schematic illustration of a light path detection module.

In the figures: 1. Instrument body, 2. Test strip, 3. Housing, 4. Test paper, 5. Display window, 6. Protective cover, 7. Chip, 8. Reaction zone, 9. Sample adding zone, 10. Marker zone, 11. Test line, 12. Quality control line, 13. Absorption zone, 14. Shell, 15. Test strip insertion port, 16. Circuit module, 17. Light path detection module, 18. Display screen, 19. Battery, 20. Light source emitting device, 21. Light receiving device.

### DETAILED DESCRIPTION OF THE PREFERRED EMBODIMENT

A clear and complete description will be made to the technical solutions in the embodiments of the present invention below with reference to the accompanying drawings in the embodiments of the present invention. Apparently, the embodiments described are only part of the embodiments of the present invention, not all of them. Based on the embodiments of the present invention, all other embodiments obtained by those of ordinary skill in the art fall within the protection scope of the present invention.

Those skilled in the art should understand that, in the disclosure of the present invention, the orientations or positional relationships indicated by the terms “longitudinal”, “lateral”, “upper”, “lower”, “front”, “rear”, “left”, “right”, “vertical”, “horizontal”, “top”, “bottom”, “inner”, “outer”, etc. are based on the orientations or positional relationships shown in the accompanying drawings, and are intended to facilitate the description of the present invention and simplify the description only, rather than indicating or implying that the device or element referred to must have a particular orientation or be constructed and operated in a particular orientation, and therefore, these terms cannot be interpreted as limiting the present invention.

It can be understood that the term “a” should be understood as “at least one” or “one or more”, that is, in one embodiment, the number of an element may be one, and in another embodiment, the number of the element may be more than one, so the term “one” cannot be understood as a restriction on the number.

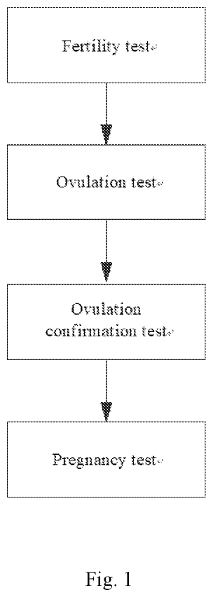

As shown in FIG. 1, a method for testing different hormones based on different time periods of pregnancy preparation includes the following steps.

In the present application, the first day of a woman's menstruation is defined as the first day of the entire menstrual cycle, which is calculated on the basis of an average women's menstrual cycle of 28 days. Menstrual days in the following descriptions are calculated on this basis.

1) Fertility test: on day 3 of the menstrual period, a subject's urine is collected as a test sample, the content of follicle-stimulating hormone in the test sample is tested with a hormone test device, and when the content of follicle-stimulating hormone in the urine is more than 15 mIU/mL, it is considered that the subject's ovarian function has damaged or the subject has entered menopause. At this time, if the subject has a pregnancy preparation plan, the subject should go to the hospital for examination in time after receiving the prompt.

The follicle-stimulating hormone (FSH) is synthesized and secreted by gonadotropic cells in the anterior pituitary gland, is used to regulate the development, growth, pubertal maturation and reproductive process, and stimulates small follicles to develop into dominant follicles in the early premenstrual period. The content level of follicle-stimulating hormone can directly reflect the functional status of the ovaries of normal women. By testing the content of follicle-stimulating hormone in the subject's urine on day 3 of the menstrual period, the ovarian functional status of the subject can be accurately reflected, the damage of ovarian function can be early warned, the pregnancy preparation women can be helped to eliminate risks in advance, and the success rate of pregnancy can be improved. In the fertility test, the subject's morning urine is used as the test sample.

Ovulation test: on day 10-12 of the menstrual period, the subject's urine is collected as a test sample, the content of luteinizing hormone in the test sample is tested with a hormone test device, and if the tested content does not reach 10 mIU/mL or more, the content of luteinizing hormone is tested again at the same time the next day until the tested content reaches 10 mIU/mL or more; then the content of luteinizing hormone is tested once every 4-6 hours until the tested content reaches 35 mIU/mL or more, indicating that an ovum will be released within 24-48 hours, intercourse is arranged at this time, and the content of luteinizing hormone in the urine is continuously tested every 4-6 hours until the tested content is restored to any value below 35 mIU/mL, intercourse is arranged again, and the test is stopped.

The luteinizing hormone (LH) is a glycoprotein gonadotropin secreted by is adenohypophysial cells, and can promote the conversion of cholesterol into sex hormones and stimulate dominant follicles to ovulate in the middle menstrual period. The content level of luteinizing hormone (LH) can be used to predict the date of ovulation. The content of LH in urine starts to increase rapidly (reach 10 mIU/mL or more) within 2-3 days before ovulation, reaches a peak (35 mIU/mL or more) about 24 hours before ovulation, and then rapidly decreases. The ovulation test is done on days 10-14 of the menstrual period. In this application, the average menstrual cycle of women is 28 days as a calculation standard. In the actual process, different women can make corresponding adjustments according to their actual menstrual days. In this step, the ovulation status of the subject is determined by analyzing the content of luteinizing hormone in the subject's urine during the menstrual period, and the intercourse time is arranged according to the ovulation status, thereby effectively improving the probability of successful pregnancy. Morning urine is usually not used as a test sample to test the content of luteinizing hormone. In the ovulation test, the content level of human chorionic gonadotropin (HCG) in the subject's urine can also be tested and combined with the content level of luteinizing hormone (LH) for comprehensive determination is and analysis.

3) Ovulation confirmation test: on at least three consecutive days within 7-10 days after ovulation, the subject's urine is collected as a test sample, the content of pregnanediol-3-glucuronide in the test sample is tested with a hormone test device, and if the contents of pregnanediol-3-glucuronide on any two consecutive days within 7-10 days after ovulation exceed 5 μg/mL, it is confirmed that the subject has ovulated; if unprotected intercourse occurs during ovulation, follow-up pregnancy test is required to determine whether the subject is pregnant. The date of ovulation can be determined by the ovulation test in step 2).

The content level of progesterone increases significantly in the late menstrual period to maintain thickening of endometrium in preparation for the implantation of a fertilized ovum, and changes in progesterone and its content level can be used to confirm whether ovulation has occurred. Take the test of PDG, one of the metabolites of progesterone, as an example: pregnanediol-3-glucuronide (PDG) is one of the metabolites of progesterone, PDG can be tested in urine, the content of PDG rises to 5 μg/mL or more within 7-10 days after ovulation and then falls back to 5 μg/mL or less, and the content of PDG will not rise to 5 μg/mL or more if ovulation does not occur in the middle period. In this is step, it can be confirmed whether the ovulation has occurred by testing the content level of metabolite pregnanediol-3-glucuronide of progesterone in urine. In this step, the content level of progesterone in the subject's urine can also be tested to confirm whether the ovulation has occurred, and the content of progesterone can also be used to evaluate whether the luteal function is normal, whether the pregnancy can be maintained, etc. In the ovulation confirmation test, the subject's morning urine is used as the test sample.

4) Pregnancy test: on day 10-14 after the ovulation, the content of human chorionic gonadotropin in a urine sample is tested with a hormone test device, and when the HCG content tested on any day of day 10-14 after the ovulation reaches or exceeds 10 mIU/mL, it can be determined that the subject is pregnant. The date of ovulation can be determined by the ovulation test in step 2).

In mature women, after the fertilized ovum moves into the uterine cavity to implant, an embryo is formed. During the process of development and growth into a fetus, the placental syncytiotrophoblast cells produce a large amount of human chorionic gonadotropin (HCG), which can be excreted in urine through the blood circulation of pregnant women. The HCG levels in serum and urine increase rapidly at 1-2.5 weeks after the fertilized ovum is formed, peaks at the 8th week of pregnancy, and then gradually decreases. In this step, the content level of human chorionic gonadotropin (HCG) in the subject's urine is tested to determine whether the subject is pregnant. In the pregnancy test, the subject's morning urine is used as the test sample. In the pregnancy test, the content levels of luteinizing hormone (LH) and follicle-stimulating hormone (FSH) in the subject's urine can also be tested and combined with the content level of human chorionic gonadotropin (HCG) for comprehensive analysis and determination.

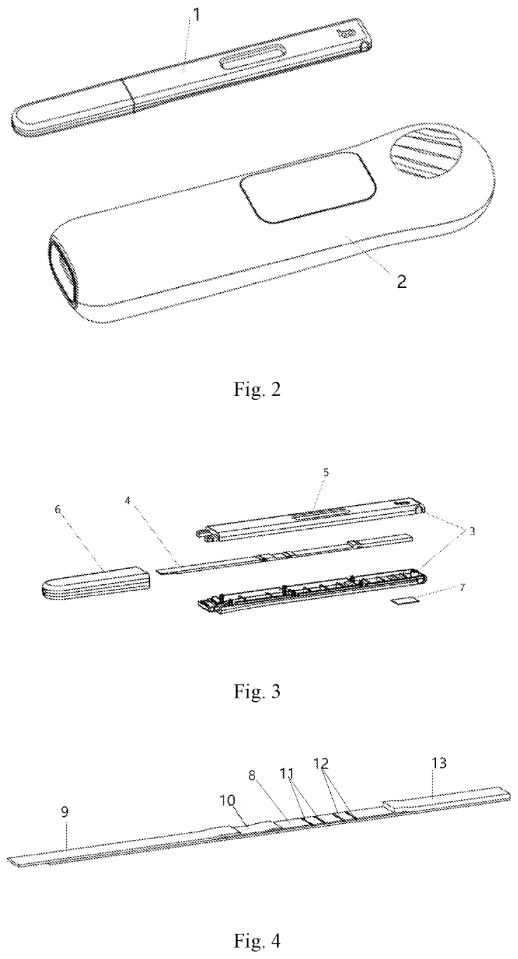

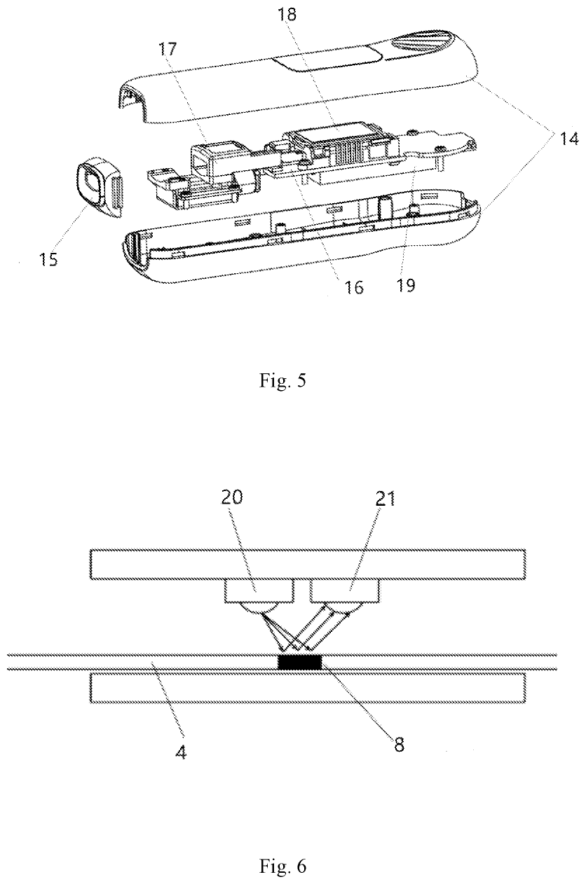

As shown in FIG. 2 to FIG. 6, the hormone test device used in the present invention includes a test strip 1 and an instrument body 2 used with the test strip. The test strip includes a housing 3, test paper 4 arranged in the housing 3, and a protective cover 6. The test paper 4 is provided with a sample adding zone 9, a marker zone 10, a reaction zone 8 and an absorption zone 13 in sequence. The sample adding zone 9 on the test paper extends out of the housing 3, and the protective cover 6 can be sleeved on the end of the housing 1 near the sample adding zone 9 to protect the sample adding zone on the test paper. Both the sample adding zone 9 and the absorption zone 13 are made of absorbing materials. The housing 1 is provided with a display window 7 corresponding to the reaction zone 8. The marker zone 10 contains one or more specific binding substances a which are marked by markers and can specifically bind to biochemical substances to be tested, the reaction zone is provided with test lines 11 corresponding to the biochemical substances to be tested, and each test line 11 is at least immobilized with one of a corresponding substance of the same type as the biochemical substance to be tested or a specific binding substance b that can specifically bind to the biochemical substance to be tested. The marker may be a fluorescent marker, colloidal gold or other types of markers. The specific binding substance a and the specific binding substance b are determined according to the type of the biochemical substance to be tested. When the biochemical substance to be tested is an antigenic substance, the specific binding substance a is a specific antibody a that can specifically bind to the antigenic substance, and the specific binding substance b is a specific antibody b that can specifically bind to the antigenic substance. The reaction zone is further provided with quality control lines 12, and each quality control line 12 is quantitatively immobilized with a specific binding substance c capable of binding and capturing the specific binding substance a. The housing 3 is further provided with a chip 7, and the chip 7 is configured to store test item information of the test strip.

The instrument body 2 includes a shell 14, a circuit module 16, a light path detection module 17, a display screen 18, and a battery 19. The shell 14 is provided with an insertion port 15 for inserting the test strip. The light path detection module 17 is electrically connected to the circuit module 16, and is configured to perform light detection on the reaction zone 8 of the test paper 4 to obtain light detection information, and to send the light detection information to the circuit module 16. The circuit module 16 processes the light detection information to obtain test results, and the display screen 18 displays the test results.

The light path detection module 17 includes at least one light source emitting device 20 and a light receiving device 21 corresponding to the light source emitting device 20. The light source emitting device 20 emits light to the reaction zone, and the light is reflected by the reaction zone and then received by the light receiving device 21. An identification module for identifying and reading the chip 8 of the test strip 1 is provided on the circuit module 16 of the instrument body 2. After the test strip is put into the instrument body, the identification module on the instrument body can read the information of the chip and give a matching test result. A wireless transmission module is further provided on the is circuit module 16 of the instrument body 2. The wireless transmission module can be a Bluetooth module, the wireless transmission module can send the data tested by the instrument body to a device such as a mobile phone, and an APP on the mobile device such as the mobile phone can display the data results; meanwhile, the instrument body has a reminding function for setting reminder time according to a user's own situation, and the instrument body can remind the user to timely test after the reminder time.

The working principle of the hormone test device is as follows: when the test strip is used, a sample is first added to the test paper, that is, the test sample is added to the sample adding zone of the test paper, the test sample containing at least one biochemical substance to be tested (the biochemical substance to be tested may be LH, FSH, HCG, PDG, etc.); the biochemical substance to be tested permeates the marker zone under the chromatographic action of the test paper, and then binds to the corresponding specific binding substance a in the marker zone to form a binding body with a marker, and the binding body continues to move to the reaction zone under the chromatographic action of the test paper; after the binding body reaches the position of a corresponding test line in the reaction zone, since the specific binding substance b immobilized on the test line can specifically bind to the biochemical substance to be tested in the binding body, the binding body will be captured and bound by the specific binding substance b immobilized on the test line; when the concentration of the biochemical substance to be tested in the test sample is higher, more marker is captured and immobilized on the test line; and the content of the marker on the test line is tested and analyzed by the light path detection module on the instrument body to obtain concentration data of the biochemical substance to be tested, so as to obtain a corresponding index. The marker may be a fluorescent marker or colloidal gold or other types of markers. Taking the colloidal gold marker as an example, the light path detection module on the instrument body can test the concentration by means of light detection. When the concentration of the antigen to be tested in the test sample is higher, the concentration of the marker immobilized on the corresponding test line is also higher. In this way, when the light path detection module performs light detection, the reflected light signal on the corresponding test line is weaker, indicating that the concentration is higher.

For a small molecular biochemical substance to be tested such as pregnanediol-3-glucuronide (PDG), a fixed quantity of the corresponding substance of the same type as the biochemical substance to be tested is is immobilized on the test line, and the working principle is as follows: when the biochemical substance to be tested is chromatographed to the marker zone, the biochemical substance to be tested will bind to the specific binding substance a marked by the marker in the marker zone to form a binding body (the quantity of the specific binding substance a in the marker zone is fixed and greater than the quantity of antigen to be tested), the remaining specific binding substance a in the marker zone does not bind to the biochemical substance to be tested and is in a free state, the free specific binding substance a will be chromatographed to the reaction zone together with the binding body, the binding body cannot bind to the corresponding substance on the test line, but the free specific binding substance a marked by the marker will specifically bind to the biochemical substance to be tested on the test line, and the more the biochemical substance to be tested in the test sample (the higher concentration of the biochemical substance to be tested in the sample), the less the marker immobilized on the test line, so the concentration of the biochemical substance to be tested in the test sample can be reflected by the quantity of the marker on the test line. When the biochemical substance to be tested is an antigenic substance, the specific binding substance a is a specific antibody a that can specifically bind to the antigenic substance, and the specific is binding substance b is a specific antibody b that can specifically bind to the antigenic substance.

Quality control lines are provided in the reaction zone of the test paper, and the quality control lines are used to determine whether the current test is valid, so as to ensure the reliability of the test results. The working principle of a quality control line is as follows: the quantity of specific binding substance c immobilized on the quality control line is fixed, so during the test process, the quantitative range of specific binding substance a that can be bound and captured by the specific binding substance c is also definite, that is, the quantitative range of marker that can be immobilized on the quality control line during the test process is also definite, and this range is a valid range; when the instrument body tests the quantity of the marker on the quality control line, if the quantity of the marker on the quality control line is within the valid range, the test data is valid; otherwise, the test result is invalid. Taking the colloidal gold method as an example, when the marker is colloidal gold, during the light detection, the larger the quantity of the marker immobilized on the quality control line, the weaker the reflected light, otherwise, the smaller the quantity of the marker immobilized on the quality control line, the stronger the reflected light; if the signal intensity of light reflected on the quality control line is within a qualified range, the test is valid, otherwise the test is invalid.

The working principle of the light path detection module is as follows: a light source emitting device and a corresponding light receiving device constitute a light path detection unit, and each light path detection unit corresponds to a test line or a quality control line on the test reaction zone. The light source emission device emits test light of a specific wavelength, and the test light irradiates the corresponding test line or quality control line in the reaction zone, and the marker on the test line or quality control line absorbs the test light and emits reflected light of a specific wavelength. The reflected light is received by the light receiving device. When the marker is colloidal gold, during this process, the larger the quantity of the marker on the test line, the weaker the intensity of the reflected light. The light receiving device tests the intensity of the reflected light. The intensity of the reflected light can reflect the quantity of the biochemical substance to be tested, then the concentration of the biochemical substance to be tested in the test sample can be calculated, and the corresponding physiological index can be obtained.

In the present invention, one or more specific binding substances a for is different biochemical substances are provided in the marker zone of the test paper, and specific binding substances b or equivalents for different biochemical substances are immobilized on different test lines in the reaction zone of the test paper, so that the test paper can test a variety of different biochemical substances at the same time, and different test lines on the reaction zone of the test paper can display the concentration information of different biochemical substances, thus realizing the function of testing multiple physiological indexes at the same time by one test device.

The biochemical items that can be tested by the hormone test device in the present invention include but are not limited to: LH, FSH, HCG, PDG, progesterone, E1G, ALB, THC, COR, PG I, PG II, FA, PRL, TSH, CRP, 25-OH-D3, SARS-CoV-2/Flu A/Flu B, PSA, NT-proBNP, CCP, HbA1c, AMH, SF, and T.

### Embodiment 2

Embodiment 2 differs from above Embodiment 1 in that: in Embodiment 2, the light path detection module includes a light source emitting device 20 and a light receiving device 21 corresponding to the light source emitting device 20, the light source emitting device 20 emits light to the reaction zone, and the light is is reflected by the reaction zone and then received by the light receiving device 21. When the light path detection module is working, the entire light path module can move along the length of the test paper. During the movement of the entire light path module, the light reminder by the light source emitting device 20 is sequentially irradiated to each test line and quality control line on the reaction zone of the test paper, and the reflected light on each test line and quality control line is also received by the light receiving device, so that test and analysis of all test lines and quality control lines in the reaction zone 8 can be realized. The remaining features of Embodiment 2 are the same as those of Embodiment 1.

The present invention is not limited to the above-mentioned best embodiments. Any person can derive other products in various forms under the enlightenment of the present invention. However, regardless of any change in shape or structure, all other technical solutions that are the same or similar to the technical solutions of the present application shall fall within the protection scope of the present invention.

1. A method for testing different hormones based on different time periods of pregnancy preparation, comprising the following steps:
1) fertility test: on day 3 of a menstrual period, collecting a subject's urine as a test sample, testing the content of follicle-stimulating hormone in the test sample with a hormone test device, and when the content of follicle-stimulating hormone in the urine is more than 15 mIU/mL, prompting damage of the ovarian function or menopause;
2) ovulation test: on day 10-12 of the menstrual period, collecting the subject's urine as a test sample, testing the content of luteinizing hormone in the test sample with a hormone test device, and if the tested content does not reach 10 mIU/mL or more, testing the content of luteinizing hormone again at the same time the next day until the tested content reaches 10 mIU/mL or more; then testing the content of luteinizing hormone once every 4-6 hours until the tested content reaches 35 mIU/mL or more, is and based on the time period of 24-48 hours after the current time as a predicted first ovulation period, prompting the subject to conceive within the first ovulation period; continuing to test the content of luteinizing hormone in the urine every 4-6 hours after the first ovulation period until the tested content is restored to any value below mIU/mL, then arriving at a second ovulation time, prompting the subject to conceive within the second ovulation period;
3) ovulation confirmation test: on at least three consecutive days within 7-10 days after ovulation, collecting the subject's urine as a test sample, testing the content of pregnanediol-3-glucuronide in the test sample with a hormone test device, and if the contents of pregnanediol-3-glucuronide on any two consecutive days within 7-10 days after ovulation exceed 5 μg/mL, confirming that the subject has successfully ovulated during the ovulation period; and
4) pregnancy test: on day 10-14 after the first ovulation time, testing the content of human chorionic gonadotropin in a urine sample with a hormone test device, and when the content tested on any day of day 10-14 after the first ovulation time reaches or exceeds 10 mIU/mL, determining that the subject is pregnant.

2. The method for testing different hormones based on different time periods of pregnancy preparation according to claim 1, wherein the hormone test device shown comprises a test strip (1), the test strip comprises test paper (4), and the test paper is provided with a sample adding zone (9), a marker zone (10), and a reaction zone (8); the marker zone (10) contains one or more specific binding substances a which are marked by markers and can specifically bind to biochemical substances to be tested, the reaction zone (8) is provided with test lines (11) corresponding to the biochemical substances to be tested, and each test line (11) is at least immobilized with one of a corresponding substance of the same type as the biochemical substance to be tested or a specific binding substance b that can specifically bind to the biochemical substance to be tested.

3. The method for testing different hormones based on different time periods of pregnancy preparation according to claim 2, wherein the reaction zone (8) is further provided with quality control lines (12), and each quality control line (12) is quantitatively immobilized with a specific binding substance c capable of binding and capturing the specific binding substance a.

4. The method for testing different hormones based on different time periods of pregnancy preparation according to claim 2, wherein the test paper is further provided with an absorption zone (13).

5. The method for testing different hormones based on different time periods of pregnancy preparation according to claim 2, wherein the marker is colloidal gold or a fluorescent marker.

6. The method for testing different hormones based on different time periods of pregnancy preparation according to claim 2, wherein the hormone test device further comprises an instrument body (2), and the instrument body is provided with a display screen (18), a circuit module (16), and a light path detection module (17) capable of performing light detection on the reaction zone of the test paper (4).

7. The method for testing different hormones based on different time periods of pregnancy preparation according to claim 2, wherein the biochemical substance to be tested is one of or a combination of luteinizing hormone, pregnanediol-3-glucuronide, follicle-stimulating hormone and human chorionic gonadotropin.

8. The method for testing different hormones based on different time periods of pregnancy preparation according to claim 2, wherein the test strip (1) is provided with a chip (7) for storing test item information, and the instrument body (2) is provided with an identification module for identifying and reading the chip (7).
